(19) United States
(12) Patent Application Publication
(10) Pub. No.: US 2017/0239175 A1
(43) Pub. Date: Aug. 24, 2017

(54) TOPICAL ANORGASMIA THERAPY

(71) Applicants: RONALD J. THOMPSON, Cincinnati, OH (US); James M. Thompson, Cincinnati, OH (US)

(72) Inventors: RONALD J. THOMPSON, Cincinnati, OH (US); James M. Thompson, Cincinnati, OH (US)

(73) Assignee: FSD Pharma Co, LLC, Cincinnati, OH (US)

(21) Appl. No.: 15/047,960

(22) Filed: Feb. 19, 2016

(51) Int. Cl. A61K 9/00; A61K 31/05; A61K 47/32; A61K 47/38; A61K 47/10; A61K 31/4985; A61K 31/198

(52) U.S. Cl. CPC A61K 9/0034; A61K 31/4985; A61K 31/05; A61K 31/198; A61K 47/38; A61K 47/10; A61K 47/32

(57) ABSTRACT

A composition and method are disclosed for treating orgasmic disorder in a female subject. The composition is a topical medicament containing tadalafil, menthol, and 1-arginine. The composition typically further comprises an extended release mucoadhesive polymer, and is used for topical application to the clitoris and genital tissues of a human female suffering from orgasmic disorder, which includes difficulty achieving orgasm and anorgasmia. Topical application of the inventive composition to the clitoral mucous membranes and other female genital tissues affects vasodilation and clitoral erection tier an extended period of time and compensates for low testosterone levels in a female subject.

### FIELD OF THE INVENTION

The present invention relates in general to a composition and method of therapy for Female Sexual Interest/Arousal Disorder (FSIAD), and in particular to a topical composition comprising the combination of menthol, 1-arginine and tadalafil for treating orgasmic disorder in a female subject.

### BACKGROUND OF THE INVENTION

Female sexual arousal disorder is defined by a complete lack of, or significant reduction in, sexual interest or sexual arousal. The problem may be lifelong or acquired, it can be the result of environmental and/or cultural factors, and its severity may fall on a continuum of mild to moderate to severe. The problem may be situational, occurring only in some instances and not others, or generalized with no apparent limitations. Personal and relationship distress can also be an important cause, making it difficult to identify clinical trial end points in the development of drugs While maintaining safety and efficacy.

Tadalafil (Cialis®), vardenafil (Levitra®, Staxin®), sildenafil (Viagra®), avanafil (Stendra®/Spedra®), udenafil (Zydena®), mirodenafil (Mvix®) and lodenafil (Helleva®) are all inhibitors of the phosphodiesterase-5 enzyme (ME-5) which destroys cyclic guanosine monophosphate (cGMP). The primary mechanism of action of PDE-5 inhibitors involves a temporary disabling of the phosphodiesterase-5 enzyme, allowing cGMP to persist. All of these drugs are able to treat male impotence caused by compromised blood flow to the penis. The Nitric Oxide Synthase biochemical pathway converts tissue 1-arginine into nitric oxide (NO) and cGMP, both potent vasodilators. The physiologic action of NO is very transient, measured in seconds, but it stimulates the production of cGMP Cyclic GMP in turn signals the smooth muscles lining the blood vessels of the penis to dilate and allow stronger blood flow. Therefore, cGMP is a key player in the erectile process.

Cyclic GMP is normally degraded by the PDE-5 enzyme. The PDE-5 enzyme inhibitors slow down the normal degradation of cGMP, and cGMP accumulates for an extended period of time, measured in hours. By holding PDE-5 at bay for 4 to 36 hours, these drugs allow cGMP to facilitate the erectile process without interference. The PDE-5 inhibitors sildenafil and vardenafil, for example, provide 4-5 hours duration of action, while tadalafil (approved for daily use) provides up to 36 hours duration.

The sildenafil/Viagra® story is one of pharmaceutical legend, Sildenafil was initially developed as an oral anti-angina medication, but in the clinical studies 80% of men reported sustained penile erections. The scientists at Pfizer, headed by Dr. John Lamattina, decided to use sildenafil as an oral therapy for impotence. The definition of “impotence” is the inability to establish and maintain a penile erection adequate for intercourse. In the 1990's there were no oral therapies for impotence. Viagra was the first. Pfizer marketers rebranded impotence as Erectile Dysfunction, and further as ED. In 1998, Viagra was introduced into the U.S. Also in 1998, the FDA first allowed direct-to-consumer advertising of prescription drugs. The combination of the first oral therapy for impotence, marketed as ED, and print and television advertising, propelled Viagra® to a multibillion dollar per year product.

U.S. Pat. No. 5,250,534 to Bell et al. (and assigned to Pfizer) titled “Pyrazolopyrimidione antianginal agents” is the original sildenafil “composition of matter” patent. This patent addresses only an antianginal use for sildenafil. U.S. Pat. No. 6,469,012 to Ellis et al. titled “Pyrazolopyrimidiones for the treatment of impotence” was filed alter the discovery of sildenafil's action as an effective impotence therapy. Both of these patents are incorporated by reference herein in their entirety. Impotence is a male-only dysfunction, and the “method of use” for the '012 patent only applies to males being treated with sildenafil.

Female Sexual Interest/Arousal Disorder (FSIAD) and the FDA: Female sexual arousal is a neurophysiologic process and is characterized by increased clitoral and vaginal blood perfusion, leading to enhanced sensation, genital swelling and vaginal lubrication. Female sexual interest/arousal disorder (FSIAD) has been reported to be quite severe for women who suffer from atherosclerosis or diabetes mellitus. FSIAD has also been called Female Sexual Dysfunction (FSD), Female Sexual Arousal Disorder (BAD), and Hypoactive Sexual Desire Disorder (HSDD), and is generally characterized by a lack or absence of sexual fantasies and desire for sexual activity FSIAD has been classified as having four separate components: (1) desire: libido disorders (decreased or absence of sexual desire/libido); (2) arousal disorders (decreased or absence of sexual arousal); (3) orgasmic disorders (difficulty achieving orgasm, and/or anorgasmia (complete lack of orgasm); and (4) sexual pain disorders (dyspareunia, painful intercourse).

In the 16 years following the FDA's publication of the 2000 Guidance for Industry on the clinical development of drug products for treatment of FSIAD, numerous pharmaceutical companies have expended hundreds of millions of dollars in search for an. FDA-approvable drug to treat FSIAD. Only recently (in 2015) was flibanserin, an oral selective serotonin reuptake inhibitor sold under the trade name Addyi®, approved for the treatment of pre-menopausal women with FSIAD. Flibanserin is a systemically absorbed serotonin uptake modulator medication (not a testosterone) initially developed as an antidepressant; however, because of flibanserin's dangers, the FDA requires a very stringent Risk Evaluation and Mitigation Strategy (REMS) that requires all prescribing physicians and dispensing pharmacists to be credentialed for flibanserin. Further, flibanserin only achieved FDA approval after two separate FDA denials. Flibanserin labeling also contains a “Black Box” warning not to take the drug with anti-fungal medications or alcohol.

Prior to Flibanserin, only two New Drug Applications for Female Sexual Dysfunction had been submitted to, accepted by, and evaluated by the FDA. Both drugs were denied approval because of safety concerns. Initially the FDA evaluated a transdermal testosterone patch, Intrinsa®, which is worn on the abdomen and delivers systemic testosterone to a woman. Since the efficacy of Intrinsa® was marginal (with one more sexually satisfying event per month than placebo) and because of safety concerns (exogenous testosterone is converted into estrogen in the female body, and excess exogenous estrogen is a risk factor for breast cancer), the FDA overwhelmingly voted against approval of Intrinsa. In December 2011, BioSante Pharmaceuticals announced the failure of phase three clinical trials of LibiGel®, a topical testosterone gel applied daily to the skin of the upper arm. The gel, which delivers a lower testosterone dose than Intrinsa®, failed to work better than placebo in boosting desire,

The demanding FDA efficacy tests for drugs for FSIAD has halted other developmental drug programs. For example, Vivus Pharmaceuticals discontinued the development of Alista®, a topical alprostadil cream applied directly to the clitoris for vasodilation, because the phase IIb clinical trials could not establish efficacy. Femprox®), owned by Apricus Biosciences, is also a topical alprostadil clitoral cream but formulated with a specific topical drug delivery vehicle. Femprox® is in development with positive clinical efficacy in 400 women. The FDA New Drug Application for Femprox® is several years away.

Why are there so few drugs FDA-approved for FSIAD after multiple drug development programs? In short, because human female sexual responses are complex. Dr. Elizabeth Kavaler, a urologist at Lenox Hill Hospital in New York City was quoted in the Associated Press after the initial flibanserin denial of FDA-approval, “It's a fairly complicated area. Unlike in men's sexual dysfunction where there is a major mechanical concern, in women there's no mechanical concern, so if she's not having a successful sex life, where is the problem?” A woman's sexual responsiveness has multiple variables, such as, age, estrogen status, testosterone status, and medications commonly used by women such as oral contraceptives and antidepressants. Antidepressants, especially serotonin reuptake inhibitors, are well known to decrease libido and sexual desire in the hypothalamus of the brain. Oral contraceptives decrease effective levels of normal free testosterone by increasing the production of sex binding globulins by the liver. Age itself does not impact a woman's sexual responsiveness, hut the normal decrease in estrogen in a woman's mid-thirties and beyond compromises sexual responses. Age also is associated with a decrease in a woman's effective testosterone level. When it comes to female androgen insufficiency and treatment of women with low sexual desire, it is important for clinicians to recognize that in normal women androgen levels decline by 50% from the early 20's to the mid 40's, and therefore age-related androgen insufficiency may occur in women's late 30's and 40's, as well as in postmenopausal women.

Testosterone and a Woman's Genital Sexual Responsiveness: Clitoral erection is a physiological phenomenon where the clitoris becomes enlarged and firm. Clitoral erection is the result of a complex interaction of psychological, neural, vascular and endocrine factors, and is usually associated with sexual arousal.

The female clitoris is the homologue of the male penis. The corpora cavernosa clitoridis is an adjoining, expandable pair of sponge-like regions of erectile tissue which contain most of the blood in the clitoris during clitoral erection. This is homologous to the corpus cavernosum penis in the male, with a recognizably similar structure. The part of the clitoris visible on the outside varies in size from a few millimeters to one centimeter and is located hidden in the upper labial fold. Any type of motion can increase blood flow to this organ, resulting in increased secretions which lubricate the vagina. During sexual arousal, for example, arterial blood flow to the clitoris is increased and trabecular smooth muscle within the clitoris relaxes, allowing blood to engorge the erectile tissues. The ischiocavernous and bulbocavernous muscles contract to compress the dorsal vein of the clitoris to stop drainage of the clitoris, trapping the blood, The corpora cavernosa form a main body that connects to the glans clitoridis. There is also a strip of erectile tissue (similar to the placement of the corpus spongiosum in males) running along the ventral surface of the corpora cavernosa main body. These female genital tissues make up the shaft, which is connected to the glans clitoridis. The tunica albuginea, a fibrous-elastic sheath, surrounds the shaft and glans clitoridis.

There are many ways to stimulate the clitoris, which ultimately leads to clitoral erection. In females, clitoral stimulation results in vasodilation and filling of the corporal shaft and glans clitoridis with blood, increased clitoral length and diameter, and increased blood flow and tumescence of associated structures such as the labia folds. There is also increased vaginal secretion and lubrication, engorgement of the vaginal wall, and increased diameter of the vaginal lumen. Thus, an agent that causes sexual arousal will also relieve conditions of a contracted vagina, such as vaginismus, vaginal dryness, and dyspareunia (difficult or painful coitus). Ultimately, appropriate integration of the sensory inputs from the nerve endings of the clitoris together with other stimuli culminates in orgasm, satisfaction, and termination of coitus. A woman typically achieves orgasm from an erect clitoris just as a man typically achieves an orgasm from an erect penis.

Female genital tissue vasodilation is afforded by cGMP from the nitric oxide synthase pathway, which has been reviewed above. In females the speed of the conversion of 1-arginine into nitric oxide and cGMP, and the efficiency of the conversion in the nitric, oxide pathway, are both dictated by testosterone levels. Testosterone is important for the female libido, sexual desire, and genital sexual responsiveness. A high testosterone titer causes the rapid conversion of 1-arginine into nitric oxide and cGMP, and a prompt clitoral erection. Research has shown that testosterone rapidly induces nitric oxide (NO) production via the androgen receptor-dependent activation of endothelial nitric oxide synthase (eNOS) in human aortic endothelial cells. it is thus apparent why pharmaceutical developmental programs have focused on exogenous testosterone as a treatment for FSIAD. Exogenous testosterone for women can improve libido and improve genital sexual responsiveness.

Phosphodiesterase-5 enzyme (PDE-5) inhibitors for Female Sexual Dysfunction: The use of sildenafil alone for treating sexual dysfunction in women has not been satisfactorily demonstrated in 2004, Pfizer issued a statement that it was abandoning research testing of sildenafil/Viagra® as a treatment for female sexual arousal disorder and would not file the drug with regulators for this indication, Pfizer stated, “Clinical studies, which began in 1996, tested sildenafil citrate on about 3000 women, and failed to show any benefit of sildenafil for women.” Further, Pfizer stated “Female Sexual Arousal Disorder is an emerging area of research and is far more complex than male erectile dysfunction.”

A recent study by H. G. Nurnberg et al, showed a complete or very significant reversal of a female's sexual dysfunction upon taking oral sildenafil one hour prior to sexual activity. In this study, eight out of the nine women required 50 mg of sildenafil by mouth, white the 9th woman required 100 mg of sildenafil. Another option for women who have serotonin reuptake inhibitor-induced anorgasmia is the use of vardenafil. As noted above, PDE-5 inhibitors such as vardenafil facilitate muscle relaxation and improve penile erection in men. However, there is much controversy about the efficiency of the drug used in the reversal of female sexual dysfunction. Vardenafil is similar to sildenafil, but is less expensive and may be covered under some insurance plans. A study by A. K. Ashton M.D. has shown that in the case of one particular woman, the effects of vardenafil as opposed to sildenafil have not only been comparable in the effectiveness, but that vardenafil is cheaper and requires a smaller dose. So far, vardenafil has been approved by the FDA only for use in men.

In light of the above, it would be advantageous to provide a new medication for treatment of Female Sexual Interest/Arousal Disorder (FSIAD), including difficulty achieving orgasm and anorgasmia. It would also be advantageous to provide a new medication for treatment of FSIAD which induces and prolongs sustained clitoral vasodilation and erection, without the administration of exogenous testosterone. It would also be useful to provide a topical clitoral formulation/composition that has minimal systemic absorption, a favorable risk/benefit ratio, and efficacy to establish significance over placebo, in order to allow FDA-approval of either a prescription or an over-the-counter drug indicated for the treatment of FSIAD.

### SUMMARY OF THE INVENTION

A first aspect of the invention relates to a composition comprising tadalafil, menthol, and 1-arginine for treatment of orgasmic disorders in a female subject, wherein the composition is topically applied to the clitoris and genital tissues of the subject, and wherein the composition compensates for low testosterone levels in the subject by inducing and prolonging sustained clitoral vasodilation and erection. The inventive composition typically further comprises an extended release mucoadhesive polymer, prepared as a topical film, cream or gel.

A second aspect of the invention is a composition comprising tadalafil at a concentration of between 0.1% and 4.0%, menthol at a concentration of between 0.1% and 2.0%, and 1-arginine at a concentration of between 0.1% and 5.0%, for treatment of orgasmic disorders in a female subject, wherein the composition is topically applied to the clitoris and genital tissues of the subject, and wherein the composition compensates for low testosterone levels in the subject by inducing and prolonging sustained clitoral vasodilation and erection.

A third aspect of the invention is a method for treating orgasmic disorder in a female subject, comprising topically administering a composition comprising tadalafil, menthol, and 1-arginine to the clitoris and genital tissues of the subject, wherein the topical application of the composition induces and prolongs sustained clitoral vasodilation and erection.

While the nature and advantages of the. present invention will be more fully appreciated, from the following detailed description, and more particularly defined by the appended claims.

### DETAILED DESCRIPTION OF TH INVENTION

Although the use of phosphodiesterase-5 enzyme (PDE-5) inhibitors to potentiate the Nitric Oxide/cGMP pathway is a recognized and approved treatment for enhancing blood flow into the penis and is commonly used in the therapy of male sexual dysfunction, there has been controversy about the efficacy of these agents in improving female sexual function. The present invention provides a topical medicament for treatment of Orgasmic disorders, which is a component of FSIAD which includes difficulty and/or lack of achieving orgasm, discussed in detail above. The inventive composition preferably contains the combination of low levels of the PDE-5 inhibitor tadalafil with menthol and 1-arginine, and is preferably formulated in combination with an extended release mucoadhesive polymer.

The specific composition of tadalafil, menthol, and 1-arginine is preferably delivered topically across the clitoral mucous membranes and other female genital tissues such as the corpora cavernosa, to affect vasodilation and clitoral erection for an extended period of tune. The inventive composition employs low concentrations of all components, especially tadalafil, to effect a woman's maximal sexual arousal and to allow a woman to achieve orgasm.

An inventive concept of the present invention is to concurrently utilize induction and prolongation of clitoral vasodilation to compensate for any endogenous level of testosterone, without the unsafe administration of exogenous testosterone. Another inventive concept is to formulate a dosage of topical clitoral menthol +1-arginine tadalafil that has minimal systemic absorption to allow FDA-approval of either a prescription, or an over-the-counter drug, indicated for the treatment of Orgasmic Disorder in females.

The inventive composition is intended to be delivered to and be absorbed into the vulva and vaginal mucous membranes, which consist of non-keratinized stratified squamous epithelium. Topical application and continued intimate contact with the mucosa of the female genital tissues to provide extended application/residence time is preferred for the inventive composition, because typically the period of time needed for maximum sexual arousal in a human female is between 3 and 20 minutes, and is dependent upon the presence of testosterone (either endogenous or exogenous). The present invention compensates for low testosterone levels in females by inducing and prolonging sustained clitoral vasodilation and erection. This is accomplished by providing the inventive composition via an extended release mucoadhesive polymer film, cream or gel.

The present invention envisions using low levels of the combination of tadalafil menthol, and 1-arginine in an extended release mucoadhesive polymer film, cream or gel which can be marketed as an over-the-counter (OTC) drug. This potential OTC drug can be specifically indicated as therapy for a woman's orgasmic disorder. Mucoadhesive polymers have been utilized in many different dosage forms in efforts to achieve delivery of drugs through the different mucosae. The term mucoadhesive is commonly used for materials that bind to the mucin layer of a biological membrane. To serve as mucoadhesive polymers, the polymers should possess some general physiochemical features such as predominantly anionic hydrophilicity with numerous hydrogen bond forming groups, and suitable surface property for wetting mucous/mucosal tissue surfaces. A number of charged or neutral polymers have been classified as bio/mucoadhesives, since they are known to bind to mucous membranes via covalent bonds.

A preferred mucoadhesive polymer for use with the inventive composition can include an extended release synthetic polymer which does not penetrate the clitoral mucous membrane, but which forms a linked hydrophilic coating as a mucoadhesive by absorbing some of the water from the clitoral mucous membrane. Diverse classes of polymers have been investigated for potential use as mucoadhesives, and may be useful as a drug delivery vehicle for the present inventive composition. These include synthetic polymers such as, but not limited to, polyacrylic acid (PAA), polyethylene oxide (Polyox), polymethacrylate derivatives, polycarbophil, poloxamer mixtures, Carbopol, hydroxy-methylcellulose, hydroxy-propylcellulose, hydroxypropylmethyl-cellulose (HPMC), and polyethylene glycol (PEG), as well as naturally occurring polymers such as hyaluronic acid and chitosan, alone or in combination. The extended release mucoadhesive action preferably keeps the inventive composition in continuous contact with the clitoral mucous membrane for 30-45 minutes, after a single manual application.

Vaginal mucoadhesive polymer films, creams and/or gels have several advantages over conventional vaginal dosage forms, which include portability, convenient application, prolonged retention time, even distribution, easy storage and improved stability of drug at harsh conditions. Since the maintenance of prolonged residence time of the tadalafil, menthol, and 1-arginine released from the mucoadhesive polymer is integral in the treatment of orgasmic disorders, vaginal mucoadhesive films (composed of, as a non-limiting example, Carbopol, HPMC and PEG) containing tadalafil, menthol, and 1-arginine are envisioned herein as a female controlled delivery system. Typically the composition is to be applied to the clitoris and genital tissues daily, and if not daily then before and during sexual experiences.

Menthol and related menthol analogs activate the cold receptor and elicit strong cooling sensations when applied to the penis or clitoris. The sensations evoked are intense, but they typically do not cause significant sexual arousal or interest. Menthol is a lipophilic mucous membrane permeation (penetration) enhancer that, for purposes of the present invention, allows the diffusion of 1-arginine and tadalafil across the clitoral mucous membrane barrier, therefore aiding to induce and prolong clitoral vasodilation of the clitoral corpus cavernosa and create a clitoral erection, Menthol is only functional for a short duration, i.e. about 1 minute to 5 minutes as a permeation (penetration) enhancer to allow other compounds (such as 1-arginine and tadalafil) to diffuse across the clitoral mucous membrane barrier. This is well explained, for example, in U.S. Pat. No. 6,702,733 to Thompson, which is incorporated herein by reference in its entirety. The use of an extended release mucoadhesive polymer in combination with the composition of tadalafil, 1-arginine and menthol lessens the intensity of the menthol, to prevent genital burning, and extends the diffusion of 1-arginine and tadalafil over a prolonged period of time, up to 20-40 minutes depending upon the molecular weight of the mucoadhesive polymer.

L-arginine excess in the clitoral corpus cavernosa induces the Nitric Oxide synthase enzyme to produce nitric oxide (NO) and cyclic GMP for induction of vasodilation. The rate limiting factor of the induction of the Nitric Oxide synthase enzyme is the availability of 1-arginine. By extending the time of the delivery of 1-arginine from the 1-5 minutes to 20-40 minutes by employing an extended release mucoadhesive polymer, clitoral vasodilation can allow a woman to achieve maximum sexual arousal and to achieve orgasm, without exogenous testosterone.

A composition of menthol+1-arginine alone acts as a Nitric Oxide synthase inducer. The menthol component causes a reflex vaginal lubrication and functions as a mucous membrane permeation enhancer to allow the 1-arginine to penetrate the clitoral mucous membrane and concentrate in the clitoral corpus cavernosa. The concentrated 1-arginine induces the Nitric Oxide synthase enzyme to produce NO. Once produced and released, NO diffuses to neighbor cells and reaches its target, soluble guanylate cyclase (sGC), The activation of sGC, caused by NO binding to its hence moiety, induces an increase in cyclic guanylate monophosphate (cGMP), a signaling messenger that activates GNP-dependent pathways. NO and cGMP are both potent vasodilators, and can act to create a clitoral erection.

The inventive composition of tadalafil+menthol+1-arginine is an improvement upon the use of the composition of menthol+1-arginine alone, as well as an improvement over the use of other PDE-5 inhibitors for treatment of female orgasmic disorder, For comparison, the molecular weights of the preferred components of the inventive composition are, menthol: 156.27 g/mol; 1-arginine: 174.20 g/mol; tadalafil: 389.60 g/mol; other PDE-5 inhibitors have a comparatively larger molecular weight than tadalafil: sildenafil: 474,00 g/mol; vardenafil: 579.00 g/mol. The lower molecular weight of tabled allows it to diffuse across the clitoral mucous membrane barrier and into the clitoral corpus cavernosa more easily than other PDE-5 enzyme inhibitors, prolonging the vasodilation initially induced by the 1-arginine.

As noted above, PDE-5 enzyme inhibitors such as tadalafil slow down the normal degradation of cGMP, such that cGMP accumulates for an extended period of time (measured in hours). Cyclic GMP signals the smooth muscles lining the blood vessels that supply the clitoris to dilate and allow stronger blood flow. This prolonged clitoral vasodilation caused by tadalafil is enhanced by its prolonged release from the extended release mucoadhesive polymer that ensures the delivery of tadalafil into the clitoral corpus cavernosa for 20-40 minutes. This can be achieved, for example, using low topical doses of tadalafil such as concentrations between 0.1% and 4.0%. Such low concentrations are significantly less than the oral concentration of tadalafil needed for ED treatment in males. Further, it is anticipated that all of the tadalafil absorption levels will be less than 10% of the oral dose. The “topical absorption of less than 10% of the oral dose” is an important FDA parameter. In addition to its lower molecular weight as compared to other PDE-5 enzyme inhibitors, tadalafil possesses other properties which make it unexpectedly useful as a topical medicament for treating female orgasmic disorders. At the time of this drafting the inventors are conducting studies to show such unexpected success. Specifically for use in treating female orgasmic disorders and compensating/correcting/accommodating for low testosterone levels in female test subjects. Thus, the combination of tadalafil (0.1%-4.0%, and preferably 1%) with menthol+1-arginine increases a woman's sexual responsiveness and a woman's ability to achieve orgasm, without administration of exogenous testosterone.

Study Design for Tadalafil+menthol+1-arginine: A blind study to test the efficacy of the inventive composition includes one Placebo Arm and Three Interventional Treatment Arms. The study is blinded and Internet based, with participants recording daily events. The Interventional Arms include: (1) Transdermal testosterone Androderm 2 mg/day, worn for 12 hours each day (therefore 1 mg/day); (2) Topical tadalafil 1-menthol+1-arginine applied to the clitoris and external genitalia daily after shower or bath and with sexual intercourse; (3) Both Transdermal testosterone Androderm 2 mg/day (worn for 12 hours each day) and use of topical tadalafil+1-menthol+1-arginine applied to the clitoris and external genitalia daily after shower or bath, and with sexual intercourse. The Placebo Arm uses a placebo transdermal patch 12 hours daily, and a placebo topical gel to he applied to the clitoris and external genitalia daily after shower or bath, and with sexual intercourse. Study Duration is planned at 16 weeks, and validated measurements include Female Sexual Function Index (FSFI) and Sexual Function Questionnaire 28 (SFQ28). Inclusion Criteria are that the participant is a female, who agrees to self attest to Orgasm Dysfunction or Anorgasmia, between the ages of 30-37 and in a stable relationship. Exclusion Criteria include a history of estrogen sensitive cancer, The endpoint is the number of sexually satisfying events per week. Statistical Analysis will include comparison of the Interventional Arm #1 to Interventional Arm #2 (i.e. direct comparison of transdermal testosterone to topical tadalafil+menthol+1-arginine); comparison of Interventional Arm #2 to Interventional Arm #3; comparison of Interventional Arm #1 to international Arm #3; and comparison of Placebo to interventional Arms #1, #2, and #3.

Menthol+1-arginine induces the clitoral corpus cavernosal Nitric Oxide synthase enzyme to produce NO and cGMP to cause clitoral vasodilation, and inclusion of tadalafil to provide the inventive composition prolongs clitoral vasodilation by inhibiting the degradation of eGMP. These are two separate and non-competing biochemical enzymatic pathways that are completely independent of one another. It is believed, therefore, that intra-cavemosal delivery of tadalafil at tissue levels approaching millimeter concentrations can cause relaxation of vascular smooth muscle and clitoral erection by a novel mechanism independent of the classical Nitric Oxide/cGMP pathway.

Menthol+1-arginine (alone) clinical trials: The combination of menthol and 1-arginine alone can induce the Nitric Oxide Synthase enzyme in women to cause clitoral corpus cavernosal vasodilation and induce a woman's sexual arousal and the ability to achieve orgasm. Two components of FSIAD were studied in this trial, sexual arousal disorder and orgasmic disorder. N=92 (multiple other proprietary and unpublished trials are comparable). Sexual arousal, as indicated by vaginal lubrication, was measured, and 86% reported an increase in lubrication rating; 78% reports d a decrease in time to maximum lubrication, Of women who were 20-39 years old, 44 of 61 reported prior inadequate lubrication, and 45% (20 of the 44) reported adequate lubrication with menthol+1-arginine. Of women who were 39-65 years old, 19 of 20 reported prior inadequate lubrication, and 60% (12 of the 20) reported adequate lubrication with menthol+1-arginine.

Orgasm was measured/determined as well, with 65% reporting increased aggregate orgasm percentage; 66% reported increased speed to orgasm; 73.5% reported increased orgasm intensity; and 60% reported increased number of multiple orgasms. Of women who were 20-29 years old, and on oral contraceptives, 96% reported orgasm with menthol+1-arginine; women 20-29 years old without oral contraceptives reported 82% orgasm with menthol+1-arginine; women 30-39 years old reported 92% orgasm with menthol+1-arginine and women 40-65 years old reported 58% orgasm with menthol+1-arginine, U.S. Pat. Nos. 6,322,493 and 6,702,733 to Ronald J. Thompson (co-inventor of the present invention), which are incorporated herein by reference in their entirety, disclose the use of a topical, clitorally-applied cream composition of menthol+1-arginine, which has been marketed under the trade name Sensua. Sensua was FDA approved as a topical medicament in 2002, as K021125.

A formulation comprising menthol+1-arginine alone does not have the efficacy to establish significance over placebo, causes genital burning in 17% of patients due to the 0.25% menthol component, and utilizes the menthol as a permeation (penetration) enhancer for 1-arginine. However, at a menthol concentration of 0.18% a formulation comprising menthol+1-arginine alone causes genital burning in only 2% of patients. A composition comprising menthol+1-arginine tadalafil utilizes menthol as a permeation (penetration) enhancer for the tadalafil. A composition comprising menthol+1-arginine+tadalafil both induces Nitric Oxide and cGMP, and prolongs the vasodilation actions of cGMP.

The present invention provides a new medication and method for treatment of difficulty achieving orgasm and/or anorgasmia which compensates for low testosterone levels in a female by inducing and prolonging sustained clitoral vasodilation and erection. Preferably the inventive topical clitoral composition has minimal systemic absorption, a favorable risk/benefit ratio, and efficacy to establish significance over placebo, in order to allow FDA-approval of either a prescription or an over-the-counter drug indicated for the treatment of FSIAD. In a preferred embodiment the inventive composition is manually applied to the clitoris and genital tissues daily, or both before and during sexual experiences. In a preferred embodiment the inventive composition is manufactured as a topically applied film, cream or gel in combination with an extended release mucoadhesive polymer.

While the present invention has been illustrated by the description of embodiments thereof, and while the embodiments have been described in considerable detail, it is not intended to restrict or in any way limit the scope of the appended claims to such detail. Additional advantages and modifications will be readily apparent to those skilled in the art. The invention in its broader aspects is therefore not limited to the specific details, representative system and method, and illustrated examples shown and described. Accordingly, departures may he made from such details without departing from the scope of the invention.

1. A composition comprising tadalafil, menthol, and 1-arginine for treatment of the orgasmic component of female sexual dysfunction, wherein the composition is topically applied to the clitoris and genital tissues of the subject, and wherein the composition compensates for low testosterone levels in the subject by inducing and prolonging sustained clitoral vasodilation and erection.

2. The composition of claim 1, wherein the composition further comprises an extended release mucoadhesive polymer as a vehicle for delivering the tadalafil, menthol and 1-arginine to the clitoris and genital tissues of the subject.

3. The composition of claim 1, wherein the tadalafil concentration is between 0.1% and 4.0%.

4. The composition of claim 3, wherein the tadalafil concentration is 1%.

5. The composition of claim 1, wherein the menthol concentration is between 0.1% and 2.0%.

6. The composition of claim 1, wherein the menthol concentration is 0.18%.

7. The composition of claim 1, wherein the menthol concentration is 0.25%.

8. The composition of claim 1, wherein the 1-arginine concentration is between 0.1% and 5.0%.

9. The composition of claim 1, wherein the 1-arginine concentration is between 2.0% and 3.0%.

10. A composition comprising tadalafil at a concentration of between 0.1% and 4.0%, menthol at a concentration of between 0.1% and 2.0%, and 1-arginine at a concentration of between 0.1% and 5.0%, for treatment of the orgasmic component of female sexual dysfunction, wherein the composition is topically applied to the clitoris and genital tissues of the subject, and wherein the composition compensates for low testosterone levels in the subject by inducing and prolonging sustained clitoral vasodilation and erection.

11. The composition of claim 10, wherein the composition further comprises an extended release mucoadhesive polymer.

12. The composition of claim 10, wherein the tadalafil concentration is 1.0%, the menthol concentration is between 0.18% and 0.25%, and the 1-arginine concentration is between 2.0% and 3.0%.

13. The composition of claim 10, wherein topical application of the composition to the clitoris and genital tissues of a human female provides minimal systemic absorption of the tadalafil, menthol, and 1-arginine.

14. The composition of claim 13, wherein the minimal systemic absorption of the tadalafil, menthol, and 1-arginine provides a favorable safety profile to enable FDA approval of the composition for orgasmic disorder therapy.

15. A method for treating the orgasmic component of female sexual dysfunction, comprising topically administering a composition comprising tadalafil, menthol, and 1-arginine to the clitoris and genital tissues of the subject, wherein the topical application of the composition induces and prolongs sustained clitoral vasodilation and erection.

16. The method of claim 15, wherein the composition is applied to the clitoris and genital tissues daily.

17. The method of claim 15, wherein the composition is applied to the clitoris and genital tissues before and during sexual experiences.

18. The method of claim 15, wherein the composition is manufactured in combination with an extended release mucoadhesive polymer.
